(19) United States
(12) Patent Application Publication
(10) Pub. No.: US 2023/0143693 A1
(43) Pub. Date: May 11, 2023

(54) IONTOPHORETIC COMPOSITION FOR ADMINISTERING S-KETAMINE

(71) Applicant: LTS LOHMANN THERAPIE-SYSTEME AG, Andernach (DE)

(72) Inventors: Michael LINN, Waldböckelheim (DE); Christoph SCHMITZ, Rheinbrohl (DE)

(21) Appl. No.: 17/908,434

(22) PCT Filed: Mar. 8, 2021
(86) PCT No.: PCT/EP2021/055734

§ 371 Date: Aug. 31, 2022

(30) Foreign Application Priority Data
Mar. 6, 2020 (DE) 10 2020 106 115.3

(51) Int. Cl. A61K 9/00; A61K 31/135; A61K 47/12; A61K 47/38; A61K 47/32

(52) U.S. Cl. CPC A61K 9/0009; A61K 31/135; A61K 47/12; A61K 47/38; A61K 47/32; A61K 9/0014

(57) ABSTRACT

The present invention relates to iontophoretic compositions comprising a ketamine salt, a thickener in an amount to provide a viscosity of at least 500 mPas (at 20° C.), and water, and to their use for treating pain or depression. The present invention further relates to kits that comprise corresponding iontophoretic compositions and an iontophoresis device.

The present invention relates to compositions and devices for transdermally administering S-ketamine. Especially, the present invention relates to compositions for iontophoretic administration of S-ketamine for the treatment of pain or depression.

### BACKGROUND

Due to the many advantages of transdermal administration in comparison to other administration routes, a variety of methods and devices have been developed for the administration of drugs through the skin.

A typical route of administration is one using passive transdermal systems (for example Transdermal Therapeutic Systems (TTS)), which deliver active substances through the skin at defined rates by diffusion processes. For certain types of drug substances, this poses the difficulty of relatively inefficient drug delivery. Ionised drugs, especially, often cannot be transported through the skin at therapeutically effective rates by the passive route.

In contrast to conventional TTS, iontophoresis is an active delivery system for active substances. The active principle of iontophoresis is based here on the fact that an electric field is generated between two electrodes placed on the skin. Depending on the loading of the active substance, the latter is present in a reservoir in the region of one of the electrodes and is repelled by the electrode closer to the reservoir and attracted by the electrode further away. In order to reach this electrode, the active substance must pass through the skin barrier and is thus absorbed by the body. However, this process only takes place as long as the electric field exists.

Iontophoresis does not necessarily have to be used with pharmaceutical active substances that have ionised functional groups, as it is also possible, for example, for the active substance to be entrained in an electrolyte-containing solution. However, the most favourable for iontophoretic administration are electrically charged active substances that are moved directly across barriers (for example the skin) within an electric field.

The process of iontophoresis was originally described by LeDuc in 1908 and even earlier in U.S. Pat. No. 222,276 (1879) and U.S. Pat. No. 486,902 (1892). Since then, iontophoresis has found commercial application in the delivery of ionically charged therapeutic drugs such as pilocarpine, lidocaine, dexamethanone, lidocaine and fentanyl.

In iontophoresis, unlike in diffusion-controlled transdermal delivery by means of TTS described above, the skin contact area of the device and the active substance concentration within the device are less important in terms of the extent to which the active substance flows through the skin. Instead, the delivery of the active substance is largely dependent on the applied current used to push the active substance into the skin.

A typical iontophoretic drug delivery system comprises an electrolytic electrical system comprising an anode and a cathode which are to be adhered to different—preferably adjacent—regions of a patient's skin, each electrode being connected by a wire to a remote power supply. Generally, this is an electrical instrument controlled by a microprocessor. Such devices, including systems with a slim design, are described, for example, in U.S. Pat. No. 5,685,837 or U.S. Pat. No. 6,745,071. Further-developed systems are also known in principle to a person skilled in the art. In addition, iontophoretic transdermal systems for lidocaine and fentanyl have been approved in the USA.

However, for many drugs, one difficulty with iontophoretic administration is that sufficient active substance must be delivered through the skin under the administration conditions to reach the “therapeutic window” of the particular active substance. For this purpose, the current density (=current flow/contact area) that can be used for the system is subject to relevant restrictions, which must not exceed a certain level above which skin irritation and burns may occur. This would strongly affect the acceptance of such an administration system.

For the treatment of depression or pain by means of S-ketamine, nasal sprays and infusions are currently used, which have a rapid uptake of the drug in the plasma. However, this is associated with side effects; in addition, the further pharmacokinetics are difficult to control. Oral thin films (OTFs) have been proposed as another alternative application form of S-ketamine. However, even with such systems it has not yet been possible to realise completely satisfactory pharmacokinetics.

An iontophoretic administration of ketamine was described in Vranken et al, Pain, 2005 (11), pp. 224-231, although only a conventional injection solution was used for administration. No improvement in pain score was observed in the studies conducted in this publication.

Against this background, there is a need for an administration system for S-ketamine that can realise the most favourable possible pharmacokinetics with the greatest possible suppression of undesirable side effects. The present invention addresses this need.

### DETAILED DESCRIPTION

In accordance with the foregoing, it has been the aim of the present invention to provide an administration system and, more especially, an iontophoretic administration system for S-ketamine which is capable of providing effective doses for the treatment of pain or depression and in which satisfactory pharmacokinetics are achieved with substantial suppression of side effects.

Accordingly, in a first aspect, the present invention relates to an iontophoretic composition containing

(i) a ketamine salt,
(ii) a thickener in an amount to provide a viscosity of at least 500 mPas (at 20° C.), and
(iii) water.

The use of a thickener to provide the minimum viscosity specified serves to establish a desired ionic strength that can improve the efficiency of the iontophoretic process such that effective amounts can be administered to treat pain and/or depression.

The active substance ketamine is present in the iontophoretic composition according to the invention as a salt and preferably as a salt of “S-ketamine” (see below), in which the amine nitrogen atom is protonated.

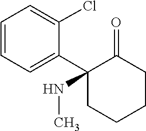 [SMILES: CN[C@]1(c2ccccc2Cl)CCCCC1=O]

Suitable anions for such salts include chloride, bromide, iodide, sulfate, phosphate, lactate, citrate, tartrate, salicylate, succinate, maleate, gluconate, mesylate, laurate, dodecylate, myristate, palmitate, stearate, coconoate, behinate, oleate, linoleate, linolenate, eicosapentaenoate, eicosahexaenoate, docosapentaenoate, docosahexaenoate, eicosanide and the like. Most preferred is the chloride salt.

The content of ketamine should expediently be such that an effective amount of ketamine can be administered over a relatively long period of time, such as 2 to 12 hours or 4 to 8 hours. For this, a sufficient amount of the active substance must be dissolved in the composition, and therefore the concentration of the ketamine must not be set too low. On the other hand, the ketamine salt cannot be dissolved in the iontophoretic composition in any quantity. A ketamine salt concentration in the range of from 1 to 10 wt. %, preferably from 1.7 to 8 wt. % and very especially preferably from 2.5 to 6 wt. %, in relation to the total weight of the composition, has been found to be especially useful.

For iontophoretic compositions, it has been found to be advantageous in terms of the achievable efficiency of administration if they contain, in addition to the ketamine, further ionically present components, such as salts. Especially, it is preferred if the iontophoretic composition according to the invention contains cations which are not due to protonated ketamine in a concentration in the range of from about 0.05 to about 1.5 mol/l, especially from about 0.1 to 1.0 mol/l, further preferably from about 0.12 to about 0.8 mol/l, and still further preferably from about 0.15 to 0.7 mol/l. The cations may be based here on dissolved pharmaceutically acceptable organic or inorganic salts (for example sodium chloride) or may be provided by protonation or deprotronation of thickeners or other additives in the composition which have corresponding groups.

Alternatively or additionally, it is preferred if the iontophoretic composition according to the invention has an ionic strength of ions not due to ketamine salts in the range of from about 0.05 to about 1.5 mol/l, especially from about 0.1 to 1.0 mol/l, further preferably from about 0.12 to about 0.8 mol/l, and still further preferably from about 0.15 to 0.7 mol/l.

Thickeners suitable for the purposes of the present invention are; for example, neutral thickeners, such as those based on polysaccharides. The term “neutral” refers to the fact that the thickeners have no ionic groups. Especially suitable neutral thickeners are, for example, cellulose derivatives, such as hydroxypropyl methylcellulose (HMPC), hydroxypropyl cellulose (HPC) or hydroxyethyl cellulose (HEC).

When using neutral thickeners, it is expedient to additionally incorporate a pharmaceutically acceptable salt in the iontophoretic composition in order to achieve a suitable ionic strength. For example, alkali salts or alkaline earth metal salts and especially alkali metal salts, expediently in the form of alkali metal chlorides, are especially preferred as pharmaceutically acceptable salt. Sodium chloride is very especially preferred as a pharmaceutically acceptable salt.

The proportion of pharmaceutically acceptable salts to be incorporated should expediently be in a range that is well tolerated by the skin, but on the other hand, where possible, should not be too high, so that competition of the salts with the active substance is limited to a tolerable level. Preferably, the salt is present in a concentration of at least 75 mmol, further preferably at least 100 mmol, and even more preferably at least 130 mmol. Additionally or alternatively, the iontophoretic composition according to the invention should, where possible, contain no more than 500 mmol, preferably no more than 300 mmol, and even more preferably no more than 200 mmol of pharmaceutically acceptable salt.

A further class of suitable thickeners are cationic thickeners, such as, especially, those which (under the conditions under which they are used) have protonated amino groups. Since the positive charges are localised at the amino groups, which are immobilised or have only reduced mobility due to the size of the thickener molecules in the composition, transport of these cationic thickeners to the skin or counter electrode is largely prevented when the composition is used in an iontophoretic administration method. Thus, a decrease in ionic strength can be prevented and the iontophoretic process can be maintained for relatively long periods of time while ensuring efficient transdermal absorption.

Especially suitable cationic thickeners are (meth)acrylate copolymers with amino groups, which may be primary, secondary and tertiary amino groups. The term (meth)acrylate copolymers refers here to copolymers formed from methacrylate monomers, acrylate monomers and mixtures of acrylate and methacrylate monomers. Preferred examples here are acrylate copolymers, methacrylate copolymers, alkylated acrylate copolymers and alkylated methacrylate copolymers. These copolymers contain two or more amino groups.

The alkyl group is preferably selected from (linear or branched) C1 to C12 alkyl groups, such as methyl, ethyl, propyl, isopropyl, or butyl. The alkylated copolymers may also include hydroxylated alkyl groups, preferably C1 to C12 hydroxyalkyl groups, such as hydroxymethyl, hydroxyethyl, or hydroxypropyl.

With respect to the amino group, the “dimethylaminoethyl” moiety is preferred as a component of the cationic thickener; such a group is present, for example, in dimethylaminoethyl methacrylate.

Very especially preferred thickeners are (meth)acrylate copolymers containing amine groups in the form of butylated and/or methylated methacrylate(s) and dimethylaminoethyl methacrylate. These preferred copolymers include the “basic butylated methacrylate copolymer” described in the European Pharmacopoeia (Ph. Eur.), the “aminomethacrylate copolymer” described in USP/NF, and the “aminoalkyl methacrylate copolymer E” “described in “Japanese Pharmaceutical Excipients”. Such copolymers are commercially available under the trade name Eudragit® (from Evonik Industries, formerly Degussa), for example as Eudragit® RL 100, Eudragit® RL PQ, Eudragit® RS 100, Eudragit® RS PQ or Eudragit® E 100. Eudragit® E 100 is a cationic copolymer based on dimethylaminoethyl methacrylate, butyl methacrylate and methyl methacrylate and is especially preferred as an amine-group-containing (meth)acrylate copolymer for use in the compositions according to the invention. The mean molecular weight Mw of this polymer is about 47,000 g/mol.

In general, however, it is possible to use any cationic thickeners as defined above in the iontophoretic compositions according to the invention, provided that they are toxicologically safe and suitable and/or approved for use in pharmaceutical products.

With regard to the proportion of the thickener in the iontophoretic composition according to the invention, the present invention is not subject to any relevant limitations, with the proviso that the proportion should be within a suitable range for the desired viscosity. If the thickener is a neutral thickener, a range of from 0.5 to 5 wt. %, and especially 1 to 3 wt. %, may be indicated for example as a suitable content. In the case of cationic thickeners, especially in the form of amine-group-containing (meth)acrylate copolymers, the proportion of the thickener or thickeners is in the range of from 1 to 25 wt. %, more preferably of from 5 to 22 wt. %, especially of from 10 to 20 wt. %, in relation to the total weight of the composition.

If the iontophoretic composition according to the invention contains a cationic thickener which has protonatable amino groups, it is further preferred if the iontophoretic composition additionally contains a carboxylic acid and especially a di- and/or monocarboxylic acid.

Suitable monocarboxylic acids include, especially, aliphatic monocarboxylic acids with an aliphatic group with up to 30 C atoms, wherein the acids can be linear or branched, saturated or unsaturated. Preferably, saturated C6 to C14 aliphatic monocarboxylic acids and especially preferably C12 to C14 aliphatic monocarboxylic acids are used. Aliphatic monocarboxylic acids which may be used in accordance with the present invention include, for example, hexanoic acid, decanoic acid, lauric acid, myristic acid, palmitic acid, caprylic acid and stearic acid; of these, lauric acid is preferred.

Suitable dicarboxylic acids are organic compounds substituted with two carboxylic acid functional groups, the compounds comprising linear, branched and cyclic compounds; these compounds may be saturated or unsaturated. For example, the compound may be a C4 to C10 and especially a C4-C8 dicarboxylic acid. Examples of such dicarboxylic acids include glutaric acid, adipic acid and pimelic acid, of which adipic acid is especially preferred.

In further embodiments, the iontophoretic composition according to the invention may comprise a combination of at least two aliphatic monocarboxylic acids or a combination of at least two dicarboxylic acids or a combination of at least one aliphatic monocarboxylic acid and at least one dicarboxylic acid.

Generally, the amount of aliphatic monocarboxylic acid(s) and/or dicarboxylic acid(s) is set to be at least sufficient to solubilise the amine groups of the cationic thickener containing protonatable amino groups and/or other components present in the composition and to produce a composition having the desired viscosity properties.

Preferably, the total amount of aliphatic monocarboxylic acid(s) and/or dicarboxylic acid(s) in the composition is in the range of from 0.1 to 15 wt. %, especially in the range of 0.5 to 10 wt. %.

According to a further embodiment, the concentration of the aliphatic monocarboxylic acid(s) is from 0.1 to 10 wt. %, preferably from 0.5 to 7.0 wt. %. According to a further embodiment, the concentration of the dicarboxylic acid(s) is from 0.05 to 6 wt. %, preferably from 2.0 to 4.0 wt. %. According to a further embodiment, the concentration of the dicarboxylic acid(s) is from 2 to 8 wt. %, preferably from 3.0 to 6.0 wt. %.

In one embodiment, the composition contains adipic acid in a concentration between 1.0 and 6.0 wt. %, or between 1.5 and 3.0 wt. %, or between 3.5 and 5.8 wt. %.

In further embodiments, the iontophoretic composition contains lauric acid as an aliphatic monocarboxylic acid, wherein lauric acid is present in a concentration of from 0.5 wt. % to 7.0 wt. %, or from 0.1 to 10 wt. %, or from 0.2 to 9.5 wt. %, or from 0.3 to 9.0 wt. %, or from 0.4 to 8.5 wt. %, or from 0.5 to 8.0 wt. %, or from 1.0 to 7.0 wt. %, or from 1.5 to 6.0 wt. %, or from 2.0 to 5.0 wt. %, or from 3.0 to 4.0 wt. %, or in a concentration of from about 3.40 wt. %, in relation to the total composition.

Water generally constitutes the main constituent in the iontophoretic composition according to the invention and is present especially in an amount of more than 50 wt. %, in relation to the total weight of the iontophoretic composition. Preferably, the iontophoretic composition contains at least 60 wt. % and further preferably at least 70 wt. % of water. The maximum amount of water is limited by the other constituents of the composition, and an expedient upper limit for the water content may be up to 96 wt. % and preferably up to 93 wt. %.

The composition according to the invention may optionally contain one or more further additives. Suitable additives include, but are not limited to, compounds selected from the group comprising solubility enhancers, skin permeation enhancers, preservatives and antimicrobials.

As used herein, the term “solubility enhancers” generally refers to compounds that can increase the solubility of the ketamine salt in the composition. This can be achieved by the additional incorporation of suitable excipients that modulate the possible interactions between the ketamine salt and the other components present in the composition.

Examples of solubility enhancers include, but are not limited to, diols such as propylene glycol and glycerol; monoalcohols such as ethanol, propanol and higher alcohols; dimethyl sulfoxide (DMSO), dimethyl formamide, N,N-dimethylacetamide and N-substituted alkyl-azacycloalkyl-2-ones. As indicated above, compounds selected from the groups of aliphatic monocarboxylic acids and dicarboxylic acids are especially effective in increasing the solubility of cationic thickeners containing amino groups.

The term “skin permeation enhancers” refers specifically to compounds that can increase the permeability of the skin to the ketamine salt contained in the composition. Due to this increase in skin permeability, the rate at which the ketamine salt can penetrate through the skin and enter the blood circulation is also increased. The increased permeation caused by the use of the skin permeation enhancers can be investigated and confirmed by measuring the rate of active substance diffusion through animal or human skin using a diffusion cell device generally known in the prior art.

Examples of skin permeation enhancers include, but are not limited to, dimethyl sulfoxide (DMSO), N,N-dimethylacetamide (DMA), decyl methyl sulfoxide (C10 MSO), polyethylene glycol monolaurate (PEGML), propylene glycol (PG), propylene glycol monolaurate (PGML), glycerol monolaurate (GML), lecithin, the 1-substituted-alkyl-azacycloalkyl-2-ones, especially 1-N-dodecylcylazacycloheptan-2-one, alcohols and the like. The skin permeation enhancer may also be selected from vegetable oils, for example safflower oil, cottonseed oil or corn oil. Combinations comprising two or more different skin permeation enhancers may also be used.

The term “antimicrobials” means agents capable of preventing the growth of microbes in a pharmaceutical preparation, especially in a composition according to the present invention. Examples of suitable antimicrobial agents include, but are not limited to, chlorhexidine salts, such as iodopropynyl butyl carbonate, diazolidinyl urea, chlorhexidine digluconate, chlorhexidine acetate, chlorhexidine isethionate, or chlorhexidine hydrochloride. Other cationic antimicrobial agents, such as benzalkonium chloride, benzethonium chloride, triclocarbon, polyhexamethylene biguanide, cetylpyridinium chloride or methylbenzethonium chloride may also be used.

Other usable antimicrobials include halogenated phenolic compounds, such as 2,4,4′-trichloro-2-hydroxydiphenyl ether (triclosan), parachlorometaxylenol (PCMX); methyl para-hydroxybenzoate; and short-chain alcohols, such as ethanol, propanol and the like.

Preferably, the total concentration of the antimicrobial(s) is in the range of from 0.01 to 2 wt. %, in relation to the total weight of the iontophoretic composition in which it is contained.

Suitable preservatives for incorporation in the iontophoretic composition according to the invention are, for example, sodium azide (NaN₃) or parabens such as para-hydroxybenzoic acid ester (Nipagin). Amounts in the range of from 0.01 to 1.0 wt. %, preferably from 0.05 to 0.5 wt. %, further preferably from 0.07 to 0.4 wt. %, still more preferably from 0.08 to 0.3 wt. %, still more preferably from 0.09 to 0.2 wt. %, and most preferably about 0.10 wt. % can be stated as a suitable proportion for preservatives, in relation to the total weight of the iontophoretic composition, with para-hydroxybenzoic acid ester being the most preferred preservative.

The invention also includes embodiments in which the iontophoretic composition is adsorbed into an adsorbent material saturated or impregnated with the composition. The adsorbent material saturated or impregnated with the iontophoretic composition serves to fix the composition while maintaining the low-viscosity structure of the composition. Suitable adsorbent materials can be selected from fibrous pads, sponges, fabrics, non-woven or woven materials, felts or felt-like materials, etc.

According to a further embodiment, the iontophoretic composition of the present invention has adhesive properties that maintain the composition in direct and complete contact with the skin throughout the period of transdermal administration at the site of application. The adhesive properties can be obtained by incorporating one or more adhesive polymers into the compositions. Adhesive polymers suitable for this purpose are generally known to a person skilled in the art. Preferably, an adhesive polymer having amine groups, which may be protonated or deprotonated, is used as the adhesive polymer(s).

In one embodiment, the iontophoretic compositions of the invention may be self-adhesive. To render the compositions self-adhesive, one or more additives selected from the group of tackifiers may be incorporated. These include, but are not limited to, hydrocarbon resins, rosin derivatives, glycols (such as glycerol, 1,3-butanediol, propylene glycol, polyethylene glycol) and succinic acid.

The iontophoretic compositions according to the invention have an electrical conductivity suitable for iontophoretic administration and a pH value at which the active substance ketamine is substantially in protonated form. With regard to the electrical conductivity, values of at least 5 mS/cm can be stated as suitable and a range of from 7 to 30 mS/cm can be stated as especially suitable. For the pH, a range of from 3.5 to 7.0 is considered preferred and a range of from 4.9 to 6.5 is considered especially preferred. Within the scope of the invention, the electrical conductivity is to be determined at 20° C. using a suitable conductivity meter.

As already apparent from the foregoing, the iontophoretic composition according to the invention also exhibits an increased viscosity which is imparted by the thickener. A range of from 600 to 2500 mPas and, especially, of from 650 to 2000 mPas can be stated as an especially suitable viscosity for the iontophoretic composition according to the invention. These viscosities are to be determined in each case at 20° C. according to (plate/cone 35 mm 1°, gap 0.05, shear rate 92.11/sec, measuring time 60 sec), wherein for example a Hacke RheoStress RS 6000 viscometer can be used.

The present invention further relates to any embodiments of the iontophoretic composition which may result from the combination of two or more of the embodiments described above, or from the combination of one or more individual features mentioned throughout the above description with any of the embodiments of the present invention described above.

As explained above, the iontophoretic composition according to the invention can be used expediently in the treatment of pain or depression. Therefore, another aspect of the present invention relates to an iontophoretic composition as described above for use in the treatment of depression or pain. Similarly, the present invention relates to the use of an iontophoretic composition as described above in a method for transdermally administering a ketamine salt, and especially an S-ketamine salt, to a patient.

Since iontophoretic compositions are regularly administered using an iontophoresis device, another aspect of the present invention relates to a kit comprising an iontophoresis device and an iontophoretic composition as described above.

In the following, the present invention will be illustrated in greater detail by means of a few examples which, however, are not to be regarded as limiting the scope of protection of the application in any way.

### Example 1

Different iontophoretic compositions were prepared with the formulations given in the following Table 1. The pH value, electrical conductivity and viscosity (each at 20° C.) were determined for these. The determined values are also given in Table 1.

TABLE 1

| Ingredient | Composition 1 | Composition 2 | Composition 3 |
|---|---|---|---|
| S-ketamine HCl | 4% | 4% | 4% |
| NaCl | 0.9% | | |
| Lauric acid | | 3.48% | |
| Adipic acid | | 2.53% | 5.63% |
| HPC (Klucel HF) | 2% | | |
| Eudragit E 100 | | 18% | 19.29% |
| Water | 93% | 71.89% | 70.98% |
| Nipagin | 0.1% | 0.1% | 0.1% |
| Conductivity | 20.6 | 8.45 | 9.24 |
| [mS/cm] | | | |
| pH | 4.39 | 5.95 | 4.97 |
| Viscosity | 1846 | 735 | 1152 |

* hydroxypropyl cellulose

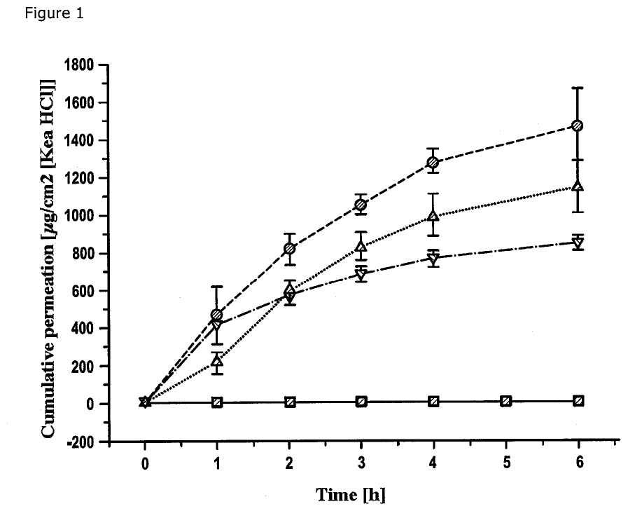

The formulations were then measured using an apparatus as described in FIG. 1 of WO2009153019, using a human skin (800 μm) and a current strength of 600 μA/cm².

The results of the measurements are shown in FIG. 1. It was found that the formulation with lauric acid (▴, composition 2) was able to achieve better cumulative permeation than the formulation containing only adipic acid (▾, composition 3). Furthermore, it was found that even higher cumulative permeation could be achieved when formulated with isotonic saline solution (•, composition 1). This is surprising because the active substance (ketamine) is in competition with Na⁺ ions in this case. Compared to the passive system (▪, composition 1 without application of voltage), permeation is significantly increased in all cases.

1. An iontophoretic composition comprising
(i) a ketamine salt,
(ii) a thickener in an amount to provide a viscosity of at least 500 mPas (at 20° C.), and
(iii) water.

2. The iontophoretic composition according to claim 1, characterised in that the ketamine salt is present in the form of an S-ketamine salt.

3. The iontophoretic composition according to claim 1, characterised in that it contains cations which are not due to protonated ketamine in a concentration in the range of from about 0.05 to about 1.5 mol/l and/or has an ionic strength, based on the ions not due to ketamine salts, in the range of from about 0.05 to about 1.5 mol/l.

4. The iontophoretic composition according to claim 1, characterised in that it contains a neutral thickener and a pharmaceutically acceptable salt.

5. The iontophoretic composition according to claim 1, characterised in that it contains a cationic thickener.

6. The iontophoretic composition according to claim 5, characterised in that it contains a pharmaceutically acceptable carboxylic acid.

7. The iontophoretic composition according to claim 5, characterised in that it contains a monocarboxylic acid.

8. The iontophoretic composition according to claim 1, characterised in that it contains at least 60 wt. % water.

9. The iontophoretic composition according to claim 1, characterised in that it has an electrical conductivity of at least 5 mS/cm.

10. The iontophoretic composition according to claim 1, characterised in that it has a viscosity (at 20° C., determined with plate/cone 35 mm 1°, gap 0.05, shear rate 92.11/sec, measuring time 60 sec) of from 600 to 2500 mPas.

11. The iontophoretic composition according to claim 1 for use in the treatment of depression of pain.

12. A method for treatment of depression of pain comprising transdermally administering the iontophoretic composition of claim 1 to a patient.

13. A kit comprising an iontophoresis device and an iontophoretic composition according to claim 1.

14. The iontophoretic composition according to claim 2, characterised in that the S-ketamine salt is in the form of S-ketamine hydrochloride.

15. The iontophoretic composition according to claim 1, characterised in that it contains cations which are not due to protonated ketamine in a concentration in the range of from about 0.15 to about 0.7 mol/l and/or has an ionic strength, based on the ions not due to ketamine salts, in the range of from about 0.15 to about 0.7 mol/l.

16. The iontophoretic composition according to claim 5, characterised in that it contains a C12 to C14 monocarboxylic acid.

17. The iontophoretic composition according to claim 1, characterised in that it contains a neutral thickener based on a polysaccharide and a pharmaceutically acceptable sodium chloride salt.

18. The iontophoretic composition according to claim 1, characterised in that it contains a (meth)acrylate copolymer thickener.

19. The iontophoretic composition according to claim 5, characterised in that it contains a pharmaceutically acceptable C4 to C8 dicarboxylic acid.

20. The iontophoretic composition according to claim 1, characterised in that it contains at least 70 wt. % water, has an electrical conductivity between 4.9 to 6.5 mS/cm, and has a viscosity (at 20° C., determined with plate/cone 35 mm 1°, gap 0.05, shear rate 92.11/sec, measuring time 60 sec) of from 650 to 2000 mPas.
